(19) United States
(12) Patent Application Publication
(10) Pub. No.: US 2015/0236353 A1
(43) Pub. Date: Aug. 20, 2015

(54) FABRICATION AND FUNCTIONALIZATION OF A PURE NON-NOBLE METAL CATALYST STRUCTURE SHOWING TIME STABILITY FOR LARGE SCALE APPLICATIONS

(71) Applicant: THE ROYAL INSTITUTION FOR THE ADVANCEMENT OF LEARNING/ MCGILL UNIVERSITY, Montréal (CA)

(72) Inventors: Jean-Luc Meunier, Montreal (CA); Dimitrios Berk, Montreal (CA); Ramona Vasilica Pristavita, Laval (CA); Dustin Binny, Montreal (CA); Norma-Yadira Mendoza Gonzalez, Cote-St-Luc (CA); Pierre-Alexandre Pascone, Montreal (CA)

(73) Assignee: The Royal Institution For The Advancement Of Learning / McGill University, Pointe-Claire, QC (CA)

(21) Appl. No.: 14/408,781

(22) PCT Filed: Jun. 27, 2013
(86) PCT No.: PCT/CA2013/050503

§ 371 Date: Dec. 17, 2014

Related U.S. Application Data
(60) Provisional application No. 61/665,566, filed on Jun. 28, 2012.

(51) Int. Cl. H01M 4/90; C07D 213/20; C30B 29/54; C07C 255/52; C30B 25/10; C30B 29/64; C07D 213/16; C07D 207/44

(52) U.S. Cl. CPC H01M 4/9083; H01M 4/9008; C07D 213/16; C07D 213/20; C07D 207/44; C07C 255/52; C30B 25/105; C30B 29/64; C30B 29/54; H01M 2008/1095

(57) ABSTRACT

A pure and crystalline single-crystal nitrogen-functionalized graphene nano-flake powder comprising from 2 atomic % to at least 35 atomic % of total functionalized nitrogen within the graphene nano-flakes is disclosed. As well, the method of producing the nano-flakes that comprises injecting a carbon source into a thermal plasma system, dissociating the carbon source into carbon atomic species, transporting the carbon atomic species through a controlled nucleation zone to produce a crystalline graphene nano-flake structure, injecting the nitrogen source into the thermal plasma system dissociating the nitrogen source into nitrogen active species, and transporting the nitrogen atomic species to contact the crystalline graphene nano-flakes to produce the crystalline nitrogen-functionalized graphene nano-flakes is also disclosed. Finally, a multilayer composite comprising a carbon substrate and a layer of crystalline nitrogen-functionalized graphene nano-flakes is also described.

### FIELD OF THE INVENTION

The present invention concerns the field of nitrogen functionalized graphene nano-sized flakes and the method of making same.

### BACKGROUND OF THE INVENTION

There is an ongoing effort to develop hydrogen fuel cells technologies for energy supply, particularly Polymer Electrolyte Membrane (PEM, also called Proton Exchange Membrane) fuel cells, as a source of electrical energy for a variety of applications, including portable devices, stationary power sources, and electric vehicles. A PEM fuel cell (PEM-FC) is made of a stack of a series of active electrochemical sources each composed of two porous electrodes separated by a solid polymer electrolyte called Nafion™. Another component in the PEM-FC is the catalyst used at each boundary between the polymer and the two electrodes. The state-of-the art catalyst materials are integrated in the PEM-FC assembly using a solvent for generating an ink that is pasted onto a carbon cloth support, this catalyst ink/carbon cloth support being integrated between the porous electrode and polymer electrolyte through compression heating of the assembly. At present, the catalyst is exclusively made of nanoparticles of platinum (Pt) and is supported by “carbon black” nanoparticles, and as such costs are high and non-noble metal replacements are being sought.

Although the activities of the state-of-the-art non-noble catalysts available are now reaching a similar order of magnitude as given by platinum [E. Proietti, et al., “Iron-based cathode catalyst with enhanced power density in polymer electrolyte membrane fuel cell”, Nature Communications, Vol. 2, p. 416, August 2011], none of these have overcome the important problem of stability in the strongly acidic fuel cell environment; they degrade rapidly in the first minutes or hours of operation.

The prior art typically uses carbon black particles in the size range from a few nanometers (nm) up to 300 nm and having roughly a spherical symmetry as a support for Pt nanoparticles having a diameter generally less than 10 nm. Similar carbon black particles of spherical symmetry are also being used in the prior art for non-noble catalysts using a metal, typically iron, dispersed at the atomic level. These carbon black particles have a percentage of amorphous structures and some amount of crystalline organized structures. The preparation steps to introduce the atomic iron catalyst in the carbon black structure use the presence of the more reactive amorphous regions in a thermo/chemical preparation method for introducing the nitrogen (N) and iron functionalities on the small crystalline zones of the carbon black particles. Amorphous structures and weakly organized small crystalline-like zones still largely dominate the final carbon black non-noble metal catalyst structure, these zones being readily eroded by the acidic media in the PEM-FC. This weak organization is seen as a main reason behind the poor stability of this type catalyst over time. The erosion process rapidly eliminates the catalytic sites present at the atomic scale, the catalytic sites being made of a particular structure of 4-6 nitrogen atoms in pyridinic bonding coordinating one iron atom on the carbon structure.

Therefore a need exists for a stable non-noble metal catalyst for Polymer Electrolyte Membrane fuel cells.

### SUMMARY OF THE INVENTION

In accordance with one aspect of the present invention, there is provided a single-crystal nitrogen-functionalized graphene nano-flake comprising from 2 atomic % to at least 35 atomic % of total functionalized nitrogen.

In accordance with one aspect of the nano-flake herein described, comprising from 5 atomic % to at least 35 atomic % of total functionalized nitrogen.

In accordance with one aspect of the nano-flake herein described, comprising from 20 atomic % to at least 35 atomic % of total functionalized nitrogen.

In accordance with one aspect of the nano-flake herein described, the nano-flake further comprises a range of pyridinic nitrogen from 10% to at least 25% as a total % nitrogen of the graphene.

In another aspect of the nano-flake herein described, the nano-flake further comprises a range of pyrollic nitrogen from 10% to at least 28% as a total % nitrogen of the graphene.

In yet another aspect of the nano-flake herein described, the nano-flake comprises a nitrogen-coordination metal selected from the group consisting of iron (Fe), nickel (Ni), cobalt (Co), titanium (Ti), vanadium (V), and combinations thereof.

In still another aspect of the nano-flake herein described, the nitrogen-coordination metal is Fe.

In still another aspect of the nano-flake herein described, comprising a stability in a polymer electrolytic membrane fuel cell of at least 100 hours.

In accordance with another aspect of the present invention, there is provided a method for producing a single-crystal nitrogen-functionalized graphene nano-flake comprising: providing a carbon source; providing a nitrogen source; injecting the carbon source into a thermal plasma system dissociating the carbon source into carbon atomic species; transporting the carbon atomic species through a controlled nucleation zone to produce single-crystal graphene nanoflakes structures; injecting the nitrogen source into the thermal plasma system dissociating the nitrogen source into nitrogen active species; and transporting the nitrogen atomic species through the controlled flow/temperature zone to contact the single-crystal graphene structures to produce the single-crystal nitrogen-functionalized graphene nano-flakes.

In yet still another aspect of the method herein described, the single-crystalgraphene from the controlled nucleation zone is deposited on a surface before contact with the nitrogen atomic species.

In yet still another aspect of the method herein described, the single-crystal graphene, is a nitrogen-functionalized graphene nano-flake comprising from 2 atomic % to at least 35 atomic % of total functionalized nitrogen of the graphene.

In yet a further aspect of the method herein described, further comprises: providing a coordination metal; injecting the coordination metal into the thermal plasma system producing an active metallic species; and transporting the active species to contact the single-crystal nitrogen functionalized graphene.

In yet a further aspect of the method herein described, further comprises adding a coordination metal selected from the group consisting of iron (Fe), nickel (Ni), cobalt (Co), titanium (Ti), vanadium (V), and combinations thereof.

In still a further aspect of the method herein described, the nitrogen-coordination metal is Fe.

In yet a further aspect of the method herein described, wherein the surface on which the single-crystal graphene is deposited is a carbon substrate.

In accordance with another aspect of the present invention, there is a multilayer composite for a polymer electrolyte membrane fuel cell, the composite comprising: a substrate and a layer of single-crystal nitrogen-functionalized graphene nano-flakes on the substrate, the nano-flakes comprising from 2 atomic % to at least 35 atomic % of total functionalized nitrogen of the graphene.

In another aspect of the composite herein described, wherein the substrate is carbon cloth or carbon fiber paper.

In another aspect of the composite herein described the substrate is a porous PEM fuel cell electrode or an electron conducting porous material.

### BRIEF DESCRIPTION OF THE DRAWINGS

Reference will be made to the accompanying drawings showing by way of illustration a particle embodiment of the present invention in which:

FIG. 1 illustrates a schematic representation of a laboratory scale conical inductively coupled thermal plasma reactor producing the nitrogen-functionalized graphene nano-sized flakes according to one embodiment of the present invention;

FIG. 2. illustrates a particle size distribution (PSD) using a Malvern Particle Sizer of the graphene nano-sized flakes;

FIG. 3 is an X-ray photoelectron spectroscopy (XPS) survey image showing identified elemental peaks, where the survey scan corresponds to a nitrogen functionalization of the GNF of 33.4 atomic % (at %) nitrogen content according to one embodiment of the present invention;

FIG. 4A is a Transmission Electron Microscopic (TEM) image of the GNFs before nitrogen functionalization according to one embodiment of the present invention;

FIG. 4B is a TEM image of the GNFs after nitrogen functionalization according to one embodiment of the present invention;

FIG. 4C is a TEM image showing further detail of the crystallinity of the GNFs before nitrogen functionalization, where the inset image displays the graphene planes showing the interplanar spacing of the GNF with 7 layers according to FIG. 4A;

FIG. 4D is a TEM image showing further detail of the crystallinity of the GNFs after nitrogen functionalization, where the inset image displays the graphene planes showing the interplanar spacing with 10 layers according to FIG. 4B, therefore the crystallinity of GNF and N-functionalized GNF is the same, as the flakes appear to show no change in size, shape, or structure;

FIG. 5 is a detailed nitrogen peak with the peak deconvolution displaying the different nitrogen species present in the GNF according to one embodiment of the present invention, from right to left: 1. pyridinic, 2. nitrile and Me-Nx or C═N groups, 3. pyrrolic, 4. graphitic/quaternary groups and, 5. pyridinic oxide or other forms of oxidized nitrogen;

FIG. 6 is a PEM-FC stability test duration graph: 100 hours, where: Voltage: 0.5 V; Back pressure: 30 psi, Feed gases: H₂—O₂; Gas feed rate: 300 std. cm³/min; Humidity: 100%; Active area: 1 cm²; Mass of functionalized GNF: 1 mg; N-Functionalization: 1.88 at %; Fe-functionalization: 0.28 at %; and

FIG. 7 is an electron micrograph of a cross-section of a layer of graphene nanoflakes (GNF) (top—white powder structure) according to one embodiment of the present invention deposited on top of the weaved carbon cloth fibers (bottom—large rod-like structure) for integration in a PEM fuel cell, where the whole assembly is made in situ in the plasma reactor in sequential steps of (1) GNF nucleation and deposition on the carbon cloth, (2)N-functionalization.

### DETAILED DESCRIPTION OF THE INVENTION

The present invention produces a stable catalyst for use in PEM-FC and forms to our knowledge the only non-noble metal catalyst structure showing both the activity and the stability for replacing Pt.

The graphene structure developed in the present invention is totally crystalline and non-porous, a single crystal, in the form of single crystal sheets and is essentially free of amorphous carbon regions and free of the significant degree of disorder required for support and generation of the catalytic sites in the prior art carbon. Furthermore, the spherical-like carbon black structures of the prior art that are composed of a mixture of amorphous/crystalline and disordered regions are essentially eliminated and replaced by fully crystalline graphene sheet-like structure. The “single-crystal” graphene herein described is significantly different from carbon blacks of the prior art, these prior art carbon blacks are strongly multi-crystalline, comprising a large number of very small crystallites separated by unorganized carbon regions.

The N and Fe sites are attached to the edges surrounding this sheet-like structure composed of 5-20 atomic planes of graphene. This structure has shown to be fully stable in the acidic environment of a PEM fuel cell during 100 hours of testing. This structure is defined here as Graphene Nano-Flakes (GNF). This GNF structure is substantially or essentially crystalline with each particle forming a single-crystal flake besides the graphene sheet-like structure, no other form of carbon involving disordered carbon organization and amorphous regions are observed. A single crystal is understood to be a crystal substantially free of grain boundaries, where the crystal lattice of the crystalline graphene herein described is continuous and unbroken to the edge of the crystal. The GNF has a nitrogen functionalization ratios of up to 30 atomic % nitrogen; preferably up to 35 atomic % nitrogen. The nitrogen functionalization ranges for the crystalline GNF described here are from 2 atomic % to at least 35 atomic %; preferably, from 5 to 35 atomic %, more preferably 10 to 35 atomic %; preferably 15 to 35 atomic %, and most preferably 20 to 35 atomic % nitrogen. The GNF is organized in sheets or planes and have widths or lateral sizes in the order of 50 nm to 100 nm where 1 nm=10⁻⁹ m.

The present invention relates first to the nitrogen- and iron-functionalized graphene nanoflake (N/Fe-GNF) structures acting as both an active and stable non-noble catalyst. The present also relates to a process to generate pure, crystalline and abundant GNF structures, which in addition is able to provide in situ within the same process the nitrogen and the iron functionalities, as well as providing the N/Fe-GNF structure as a deposit made directly on the PEM-FC support when used in such an application.

The present invention uses a synthesis route where gas precursors (methane or any other light hydrocarbon molecule (gaseous or liquid), argon, and nitrogen) injected in a thermal plasma environment are used to nucleate and control the formation of a new and specific nanometer scale structure for the support of metal atoms.

This produced nitrogen-functionalized graphene nano-sized flake material has now proven to be active and stable in a PEM-FC environment, showing no loss of activity in 100-hour tests. Key aspects for both the activity, and more so for the stability, of the catalyst are first a purity of the carbon support and the achievement of a strongly crystalline structure (i.e. a minimization of the strongly reacting amorphous (non-organized) structures of the carbon material).

The synthesis process for this GNF structure also results in unique properties providing additional advantages to both the structure and the cost of fabrication. These advantages include: the carbon crystalline nano-powders generated are pure; no presence of contaminants for the fuel cell operation which eliminates possible poisoning effects, and no need for a post-cleaning step; the process generates a very narrow size distribution of the nano-powders both in the thickness (5-20 atomic planes) and in the sheet lateral sizes (may be in the order of 50 nm to 100 nm; 1 nm=10⁻⁹ m). The purity and homogeneity are requirements for the control of the catalyst structure and behavior in the PEM-FC environment. Furthermore, the process behind the present invention enables the functionalization step incorporating nitrogen atoms into pyridinic and pyrrolic sites of the GNF sheet edges to occur in situ within the same reactor. This is an advantage, as nanoparticle handling is difficult and costly; the process eliminates an important sequence of chemical and physical steps used in the current state-of-the-art carbon black functionalization. Another advantage is the amount of nitrogen incorporated in the GNF structure is the highest ever attained on any carbon support, doubling the state of the art value previously obtained [E. Proietti, et al., “Iron-based cathode catalyst with enhanced power density in polymer electrolyte membrane fuel cell”, Nature Communications, Vol. 2, p. 416, August 2011]. This material has the potential to achieve the largest activity of any of the non-noble catalysts ever developed and would substantiate the effectiveness of the N-functionalized sheet edge structure generated.

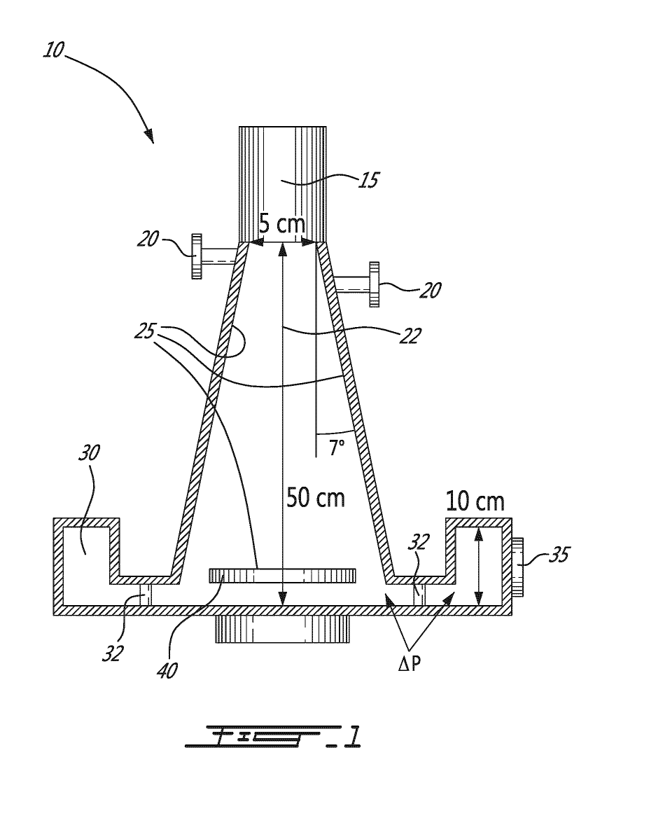

GNF Reactor 10: In the demonstration experiments, the GNF material is produced using a thermal plasma, preferably an inductively coupled thermal plasma (ICP) torch system of 35 kW nominal power, attached to a water-cooled reactor, schematically illustrated in FIG. 1. The process is readily scalable to higher power and production levels as the flow/energy fields and geometry for generating the GNF are modeled and can be scaled easily. The reactor 10 may include sight glasses 20 near the exit of the ICP plasma torch assembly 15.

All the gases are added in an ICP Plasma torch assembly 15, including as many as three flow injection sections (not illustrated): a sheath section, a central section and a probe section. Argon, nitrogen, hydrogen and other gases can be added to the sheath and central flow zones while methane, argon, nitrogen and/or other gases are typically added to the probe flow depending on what step of the GNF generation/functionalization is occurring. The central gas constitutes the plasma and the sheath gas provides cooling to the torch components. The probe flow carries gases for use in the dissociation/chemical reactions. In the present method, methane/argon are first injected in the probe to create the crystalline GNFs and then nitrogen is injected in the probe to functionalize the GNFs with nitrogen. The present reactor is a batch type reactor, but two or more reactors in series can clearly be envisaged.

Step 1: Graphene Nano-Sized Flakes Production:

Methane gas is used as the carbon precursor with a typical range of flow rates between 0.5 and 5 slpm (standard liters per minute) and preferably from 0.5 to 3 slpm. The methane is injected axially (along the axis 22 of the reactor 10) in the core of the plasma within the ICP plasma torch assembly 15, is dissociated into the carbon atomic species by the high plasma temperature, and transported downstream into the reactor. In the demonstration experiments, the power may typically be varied between 10 and 20 kW, while the reactor pressure may vary in the range between 5 to 90 kPa, and up to 55-90 kPa. Argon is injected at flowrates from 5 to 100 slpm.

Atomic carbon species are transported downstream into the reactor zone where they nucleate through a homogeneous nucleation process into solid single-crystalline graphene particles and are transported by the gas flow to the conical walls of the ICP reactor 10 and/or onto a deposition plate 40 opposite the ICP plasma torch assembly 15. A key feature of the present invention is precise control of the nucleation zone by controlling the flow stream and the temperature fields, eliminating the presence of impurities and enabling fully crystalline structures. Surprisingly, and under the well-controlled flow/energy/nucleation field conditions, the carbon nanoparticles nucleating homogeneously do not show the expected spherical geometry but rather they exhibit crystalline sheet-like structures having 50-100 nm in-plane sheet sizes and 5-20 graphene layers.

To ensure control of the nucleation zone, the ICP torch is attached to an axisymmetric conical water-cooled reactor (in one laboratory scale embodiment having a length of 50 cm and a precise angle of expansion of 14 degree angle). FIG. 1 illustrates a schematic representation of the conical reactor 10 used to prepare the nitrogen-functionalized GNF with the angle adapted to the plasma torch flow/power characteristics. Water cooling is not illustrated in FIG. 1, but generally comprises the cooling of the reactor walls that are jacketed with water flow inside.

The angle of the reactor can vary between 2° and 12° from the axis 22 of the reactor 10 or as illustrated from the vertical plane. In a preferred embodiment, this angle was found to be 7° from the axis of the reactor. The reactor 10 geometry is completely axi-symmetric and adjusted to totally eliminate any flow recirculation zones within the reactor. The flow fields are laminar and the flow and temperature fields are adjusted in this geometry to push the carbon nanoparticle nucleation fields, occurring between 3000 K and 5000 K, well downstream into the reactor and far from the fluctuating zones around the plasma torch outlet. This creates a controlled nucleation zone where a large volume of uniform nucleation occurs, enabling a precise control of the homogeneous nucleation sequence. This leads to the “robustness” of this new process, the term robustness meaning a product yield and purity that stays invariant over important variations of the plasma torch power or pressure in the reactor. Computational fluid dynamics (CFD) modeling was used to adapt the reactor geometry in order to position the nanoparticle nucleation field and enable a control of the homogeneous nucleation time scales and associated temperature/flow/chemical fields. [R. Pristavita, et al., “Carbon Nanoparticle Production by Inductively Coupled Thermal Plasmas: Controlling the Thermal History of Particle Nucleation”, Plasma Chem. Plasma Process (2011) 31:851-866]. A variation of the angle of the reactor in the given range also allows additional control over the position of the nucleation zone and the size of this nucleation zone. The position of the nucleation zone in turn allows a control over the carbon atomic precursor density which is responsible for the thickness of the GNF produced. The size of the nucleation zone in turn allows a control over the residence time of the carbon precursor inside the nucleation zone which is responsible for the length of the GNF produced. The angle of the reactor hence provides a control over the geometry of the GNF structure through independent processes modifying the thickness and the length of the graphene sheet structure.

The exhaust gases flow radially through a 4-mm gap produced by little pins 32 around the circumference of the bottom plate ensuring there is an axisymmetric radial exhaust flow, into an annular manifold 30, and out a gas exit 35. The flakes deposit as a black powder aggregate film on the reactor sidewalls and bottom deposition plate 40.

Stable temperature/velocity fields are produced in the controlled nucleation zone and form a well-defined homogeneous carbon nanoparticle nucleation field. The extremely uniform residence time distribution generated within this nucleation field yields the clean product with a narrow size distribution. The carbon nanoparticle structure observed with this “tuned” reactor showed a very different structure from the carbon black/carbon soot structures observed in all other reactor geometries previously used. The structure corresponded to the GNF structure and was observed to be in the shape of single crystals of pure, uniform and non-agglomerated graphene flakes showing important open porosity when accumulating on a substrate. This was unexpected, as no theoretical or modeling work exists at present, that predicted such a non-spherical structural evolution from homogeneous nucleation theories leading to the 2-dimensional structures observed.

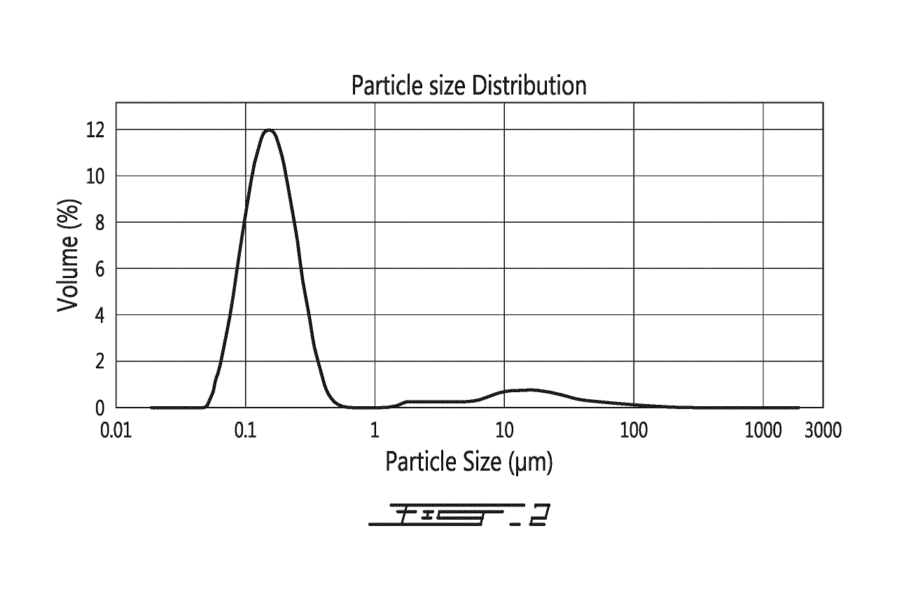

The graphene nano-sized flakes produced have a narrow particle size distribution as illustrated in FIG. 2. Particle size distribution (PSD) was obtained using a Malvern Particle Sizer. It is important to note here that this PSD measurement assumes spherical particles in the data treatment by integrating all light scattering information, regardless of the orientation of the particle. It cannot provide information for the non-isotropic 2-dimensional structure of the GNF. It, however, indicates a very narrow PSD distribution corresponding to an average graphene sheet plane dimension located between 100 and 200 nm (0.1-0.2 μm).

Step 2: Nitrogen Functionalized Graphene Nano-Sized Flakes Production:

Nitrogen is introduced into the crystalline nano-sized graphene in the following in situ method that increases the total nitrogen functionalization, and the pyridinic and pyrrolic nitrogen functionalization, to values that have never been reached before with conventional carbon based nanoparticles.

A 2-step sequence may be performed inside the reactor using two different plasma compositions, the overall sequence being performed in one batch operation in the demonstration test experiments, however a continuous process having two or more ICP system in series can clearly be envisaged where deposited crystalline graphene from a first reactor is transported to a second reactor where nitrogen functionalization occurs. As indicated above, the first step, called the “Nucleation” phase, generates the crystalline GNF structures which are deposited on the powder collecting regions 25 shown in FIG. 1, along the conical walls of the reactor and on the deposition plate opposite the ICP plasma torch assembly.

Once a substantial amount of GNF powders is made available in the reactor (typical example: 200 mg of GNF during a “Nucleation” phase of 10 minutes in the test reactor), the methane flow is stopped and the second step, called the “Functionalization” step, starts by changing the plasma gas composition to 10 to 100 slpm, and preferably 45-80 slpm, N₂, and 5 to 100 slpm, and preferably 15-30 slpm, Ar. The pressure of the reactor may vary in the range between 0.5 to 13 psia (3 to 90 kPa), preferably 1.6-8 psia (11 to 55 kPa) and up to 8-13 psia (55 to 90 kPa).

This nitrogen functionalization step is typically performed during a period of 5-30 minutes at a pressure varying between 0.5 to 10 psia (3 to 69 kPa). The nitrogen functionalization step enables excited and ionic nitrogen active species to pass through a controlled flow/temperature zone and be available downstream of the plasma zone reaching the GNF particles that have been deposited on the reactor walls and the deposition plate. The particular structure of the GNF with the graphene edge sites all around the flakes make them particularly reactive for the introduction of nitrogen, the levels of nitrogen functionalization GNF attaining up to 35 atomic %.

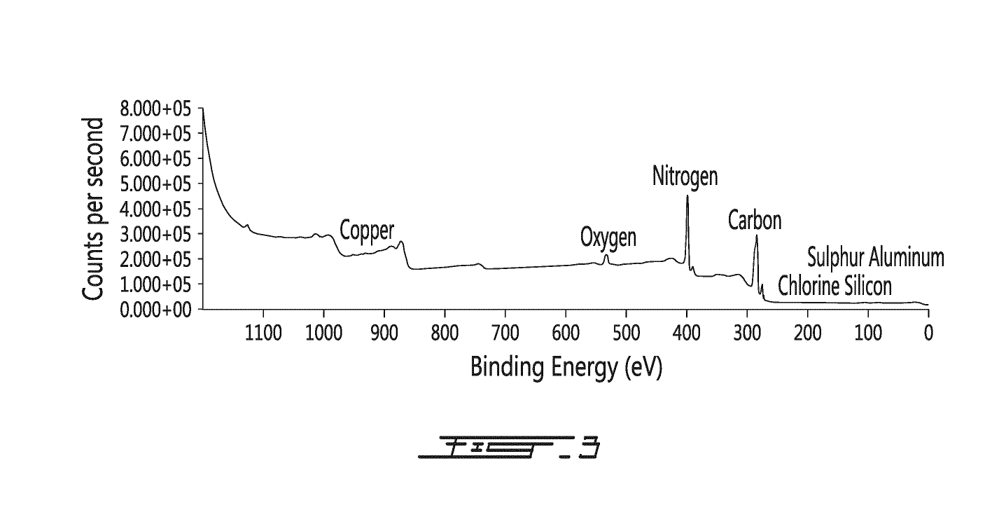

As illustrated in the X-ray photoelectron spectroscopy (XPS) spectrum of FIG. 3, 33.4% atomic % N is seen, which gives an N/C atomic ratio of 55.7% when consideration is made of the oxygen absorbed by the powders after being taken out of the reactor. Furthermore, the number of pyridinic nitrogen sites is greater than 10% of the total sites in the functionalized nitrogen. In FIG. 3 for the nitrogen-functionalized GNF, 24.4% of the N is as pyridinic sites. Therefore, from 10 to 25% of the nitrogen functionalized in the GNF produced is as pyridinic sites.

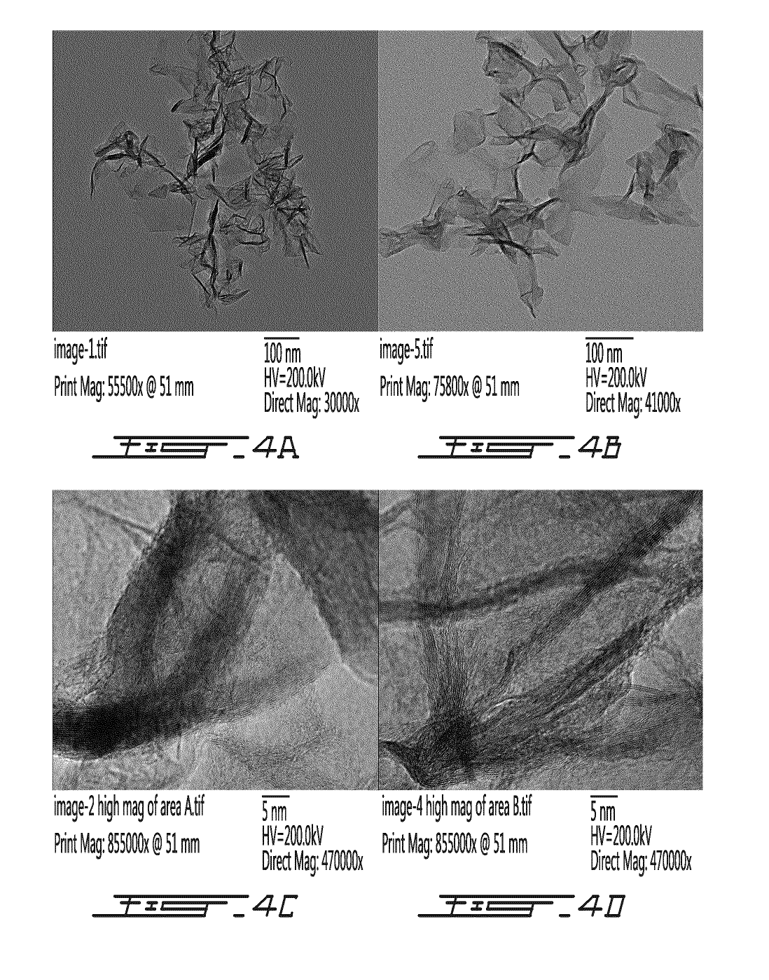

The produced nitrogen-functionalized graphene nano-flakes are illustrated in FIGS. 4A to 4D, which are Transmission Electronic Microscopic (TEM) images of the GNFs A) before and B) after nitrogen doping. Note that the flakes appear to show no change in size, shape, or structure. Further detail of the crystallinity of the GNFs is offered both in FIG. 4C before and FIG. 4D after nitrogen doping. The inset images of each display the graphene planes showing the interplanar spacing to be the same. FIG. 4C shows a GNF with 7 layers while FIG. 4D displays one with 10 layers.

The single crystals of nitrogen-functionalized GNFs of the present invention have a flake-like morphology, with a typical planar length in the order of 100 nm, and a low tendency for agglomeration resulting in an open porosity. The open porosity is believed to be important for achieving high catalytic activity, particularly in PEM-FC applications, as it is believed to facilitate the transport of the reaction product/precursor to/from the catalytic sites.

The large nitrogen integration is believed to be due in large part to the high reactivity of the carbon atoms at the graphene edges that create an ideal substitution opportunity for the nitrogen to assimilate. There is also some speculation that tortuosity in the graphene planes in the graphene matrix may lead to higher reactivity and more opportunities for nitrogen substitution.

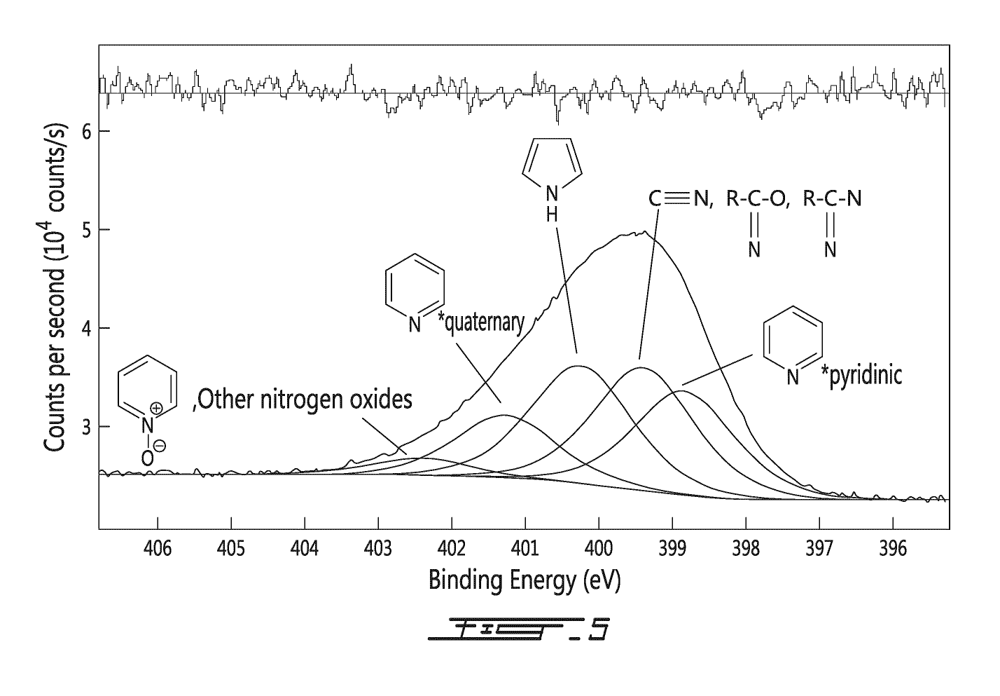

Through detailed peak deconvolution, shown in FIG. 5, one can estimate the amounts of the various nitrogen chemical structures in the GNF powder displaying the different nitrogen species present, from right to left: 1. pyridinic, 2. nitrile and Me-Nx or C═N groups, 3. pyrrolic, 4. graphitic/quaternary groups and, 5. pyridinic oxide or other forms of oxidized nitrogen. This information is also presented in Table I.

TABLE I
NITROGEN SPECIES DECONVOLUTION DERIVED FROM
XPS SPECTRA

| | | | | Common Peak |
|---|---|---|---|---|
| | Peak | Atomic | | Position |
| | Position | Percentage | Percentage of | Ranges in |
| Species | (eV) | (at. %) | Total Nitrogen | Literature (eV) |
| Pyridinic | 398.8 | 8.2 | 24.4% | 397.0-399.5 |
| Nitrile/Me—Nx/ | 399.4 | 9.6 | 28.8% | 399.0-400.5 |
| C═N groups | | | | |
| Pyrrolic | 400.2 | 9.3 | 27.9% | 399.8-401.2 |
| Graphitic/ | 401.2 | 4.9 | 14.7% | 401.0-403.6 |
| Quaternary | | | | |
| Pyridinic | 402.4 | 1.4 | 4.1% | 402.0-405.0 |
| Oxide/Other | | | | |
| Oxidized | | | | |
| Nitrogen | | | | |

The ranges for pyridinic nitrogen vary from 10% to at least 25% of the total nitrogen in the graphene. The range of pyrrolic nitrogen varies from 10% to at least 28% of the total nitrogen in the graphene.

The 2-step sequence provides an optimal and independent control of both sequences and maintains the pure and highly crystalline GNF structure while achieving the highest N-functionalization observed to date. This is made without opening the reactor and being faced with nanoparticle handling, together with complex additional chemical functionalization steps and further possibilities of material contamination.

Optionally and in a similar way, the present invention can be further functionalized with a 3-step in situ process to reach the full ready-to-use catalyst structure, these three steps being: 1. Nucleation; 2. N-Functionalization, and 3. Metallic functionalization.

Step 3: Metal-Functionalization of N-Functionalized GNF.

The nitrogen functionalized GNF inside the reactor is further reacted. The plasma conditions are modified to inject argon plus a metallic precursor, in a preferred embodiment, ferrocene (Fe(C₅H₅)₂), into the plasma flow stream to allow stoichiometric quantities of a metal such as Fe to functionalize on nitrogen-coordinating sites. The high temperature zone of the plasma breaks down the metallic precursor and allows the formation of Fe+ and excited neutral Fe species. These species will travel through a controlled flow/temperature zone that enables the transport of these species to the crystalline nitrogen-functionalized GNF for addition to the host N sites. The gas pressure and flow conditions are adapted to allow the transport of these active species to the GNF before any significant nucleation of Fe into metal particles. A variety of catalytic metals can be used to coordinate with the nitrogen of the GNF, these metals are called coordination metals or nitrogen coordination metals. The possible metals are selected from the group consisting of Fe, Ni, Co, Ti, V, and combinations thereof.

The process of metal coordination may also be conducted by including the coordination metal directly in the second step with the nitrogen plasma.

Stability Analysis:

The activity and stability demonstration experiments were performed on a PEM-FC test bench based on 1 cm² of active surface area, and using pure hydrogen and oxygen as feed gases. Tests were performed on GNF samples from the reactor having N-functionalization ratios of 1.88 at %, with 0.74 at % (39.4% of total N) of these being in a pyridinic structure. The state-of-the-art technique described by Proietti et al. was used in these tests to add the Fe-functionalization to the N-functionalized GNF structures. The resulting iron content was 0.28 at %, a very small value with respect to the activity that can be attained, but enabling a stability evaluation and comparison of the N-functionalized GNF structures with state-of-the-art catalysts and techniques. The N/Fe-functionalized GNF catalyst was then prepared with Nafion™ for deposition on the carbon cloth support and integrated in the PEM fuel cell stack. The GNF-based catalyst was used at the cathode for the oxygen reduction reaction which constitutes the most stringent requirements in terms of stability and is by far the site containing the largest amount of Pt in the conventional scheme. The typical platinum catalyst was used on the anode electrode.

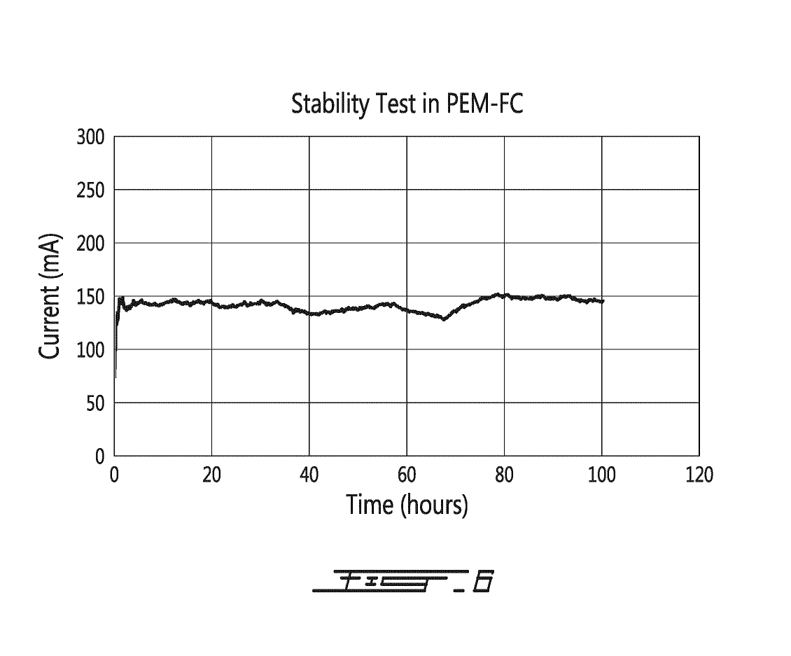

These tests performed at 0.5 V proved for the first time the full stability of a non-noble catalyst over 100 hours; i.e. showing no loss of activity during this time scale. FIG. 6 shows the evolutions of the PEM-FC activity over time in the stability test. It is to be noted that the activity value in the order of 150 mA/cm² is relatively low, which is expected since the catalyst used for these preliminary tests had low percentages of N- and Fe-functionalization.

Integration of all the synthesis steps directly onto the PEM-FC support structure: the three steps discussed above eliminate a long sequence of physical unit operations (such as ball milling) and chemical treatments in the state-of-the-art processing. The following state-of-the-art steps of integration in the fuel cell are typically made by first dispersing the carbon-based catalyst structure in a solvent using sonication to limit agglomeration. This produces an ink that is dispersed onto a carbon fiber cloth acting as the catalyst support in the fuel cell. This carbon cloth is then impregnated with Nafion™ polymer and finally pressed with a Nafion™ membrane for integration between the porous electrodes. The prior art process sequence has disadvantages that produce agglomeration of the particles, that reduce the availability of catalytic sites, modify the structure of the nanoparticles, and tend to generate poor spatial distributions of the nanoparticles because of the fluid dynamics and solvent evaporation processes. The prior art process includes other complex manipulation steps that add to operating cost and which require specific handling and disposal procedures.

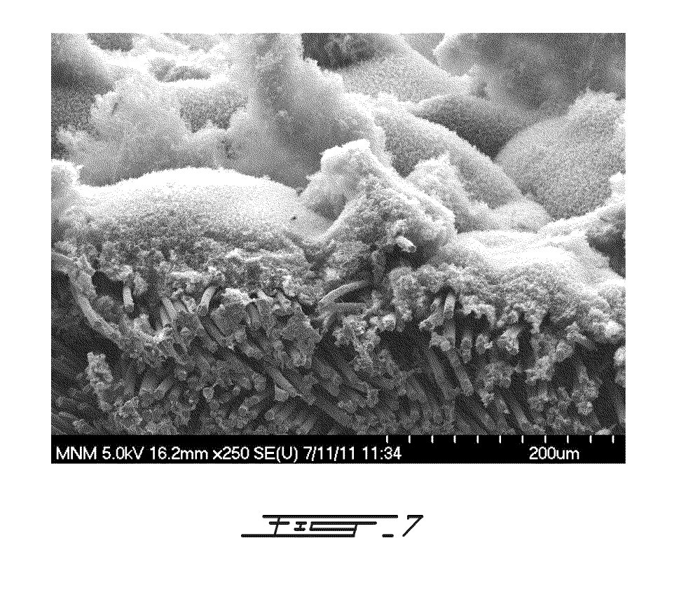

The present process eliminates most of the steps required by prior art processes, and in particular eliminates the fabrication of an ink using a solvent. Steps 1, 2, and 3 are made directly inside the reactor on the carbon fiber cloth in one processing sequence. The control of the fluid dynamics in the reactor provides for the transport and deposition of the nucleated GNF on a receiving plate. The immobilized GNF particles are then functionalized in Step 2 and 3. The PEM-FC carbon cloth support can thus be inserted directly on the receiving plate and act as a support for further functionalization and transport of the catalyst layer for Nafion™ impregnation and integration in the fuel cell. No handling of nanoparticles is necessary in the invention, as the catalyst nanoparticles are immobilized on their final support before leaving the reactor. Demonstration experiments were made under this geometry; the results shown in FIG. 7 indicate a very good layer of as-produced GNF which is well-immobilized on the carbon cloth support. This means the catalyst structure maintains its very open porosity by avoiding the steps inducing agglomeration, an added benefit for improved activity in the PEM fuel cell stack.

FIG. 7 is an electron micrograph of a cross-section of a graphene nanoflake (GNF) layer (top—white powder structure) deposited on top of a carbon substrate that in a preferred embodiment is a woven carbon cloth fiber (bottom—large rod-like structure) for integration in a PEM fuel cell. The whole assembly is made in situ in the plasma reactor using only the sequential steps of (1) GNF nucleation and deposition on the carbon cloth, and (2)N-functionalization. Other potential carbon substrates are carbon cloth or carbon fiber paper. The carbon-based substrate can also be replaced directly by one of the porous PEM fuel cell electrode receiving the functionalized GNF layer, or by another electron conducting porous material acting as an intermediate layer between the electrode and the polymer electrolyte in the PEM fuel cell assembly.

The embodiments of the invention described above are intended to be exemplary. Those skilled in the art will therefore appreciate that the foregoing description is illustrative only, and that various alternate configurations and modifications can be envisaged. Accordingly, the present invention is intended to embrace all such alternate configurations, modifications and variances which fall within the scope of the appended claims, that should not be limited by the preferred embodiments set forth in the examples, but should be given the broadest interpretation consistent with the description as a whole.

### REFERENCE

[1] E. Proietti, et al., “Iron-based cathode catalyst with enhanced power density in polymer electrolyte membrane fuel cells,” Nature communications, vol. 2, p. 416, August 2011.

1. A single-crystal nitrogen-functionalized graphene nano-flake comprising from 2 atomic % to at least 35 atomic % of total functionalized nitrogen.

2. The nano-flake of claim 1, comprising from 5 atomic % to at least 35 atomic % of total functionalized nitrogen.

3. The nano-flake of claim 1, comprising 20 atomic % to at least 35 atomic % of total functionalized nitrogen.

4. The nano-flake of claim 1, further comprising a range of pyridinic nitrogen from 10% to at least 25% as a total % nitrogen of the graphene.

5. The nano-flake of claim 1, further comprising a range of pyrollic nitrogen from 10% to at least 28% as a total % nitrogen on the graphene nano-flake structures.

6. The nano-flake of claim 1, comprising a nitrogen-coordination metal selected from the group consisting of Fe, Ni, Co, Ti, V, and combinations thereof.

7. The nano-flake of claim 6, wherein the nitrogen-coordination metal is Fe.

8. The nano-flake of claim 1, comprising a stability in a polymer electrolytic membrane fuel cell of at least 100 hours.

9. A method for producing a single-crystal nitrogen-functionalized graphene nano-flake comprising:
providing a carbon source;
providing a nitrogen source;
injecting the carbon source into a thermal plasma system dissociating the carbon source into carbon atomic species;
transporting the carbon atomic species through a controlled nucleation zone to produce a single-crystal graphene;
injecting the nitrogen source into the thermal plasma system dissociating the nitrogen source into nitrogen active species; and
transporting the nitrogen atomic species through the controlled flow/temperature zone to contact the single-crystal graphene to produce the single-crystal nitrogen-functionalized graphene nano-flake.

10. The method of claim 9, wherein the single-crystal graphene from the controlled nucleation zone is deposited on a surface before contact with the nitrogen atomic species.

11. The method of claim 11, wherein the single-crystal graphene is a nitrogen-functionalized graphene nano-flake comprising from 2 atomic % to at least 35 atomic % of total functionalized nitrogen of the graphene.

12. The method of claim 9, further comprising:
providing a coordination metal;
injecting the coordination metal into the thermal plasma system producing an active metallic species;
transporting the active metallic species to contact the single-crystal nitrogen-functionalized graphene; and
producing a nitrogen-functionalized graphene nano-flake comprising metal.

13. The method of claim 9, further comprising adding a metal selected from the group consisting of Fe, Ni, Co, Ti, V, and combinations thereof.

14. The method of claim 12, wherein the nitrogen-coordination metal is Fe.

15. The method of claim 7, wherein the surface on which the single-crystal graphene is deposited is a carbon substrate.

16. A multilayer composite for a polymer electrolyte membrane fuel cell, the composite comprising:
a substrate and
a layer of single-crystal nitrogen-functionalized graphene nano-flakes on the substrate, the nano-flakes comprising from 2 atomic % to at least 35 atomic % of total functionalized nitrogen of the graphene.

17. The composite of claim 16, wherein the substrate is carbon cloth or carbon fiber paper.

18. The composite of claim 16, wherein the substrate is a porous PEM fuel cell electrode or an electron conducting porous material.
